(19) United States
(12) Patent Application Publication
(10) Pub. No.: US 2021/0065866 A1
(43) Pub. Date: Mar. 4, 2021

(54) SMART MEDICATION CONTAINER

(71) Applicant: Tech Pharmacy Services, LLC, Fort Lee, NJ (US)

(72) Inventors: Omer EINAV, Kfar-Monash (IL); Doron SHABANOV, Tzur-Yigal (IL); Tamir BEN DAVID, Tel-Aviv (IL); Anthony Joseph SPERO, Queensbury, NY (US); Eyal LIVSCHITZ, Givat Shmuel (IL); Thomas A. MCKINNEY, Boonton, NJ (US); Moshe LIBERMAN, Yehud (IL)

(73) Assignee: Tech Pharmacy Services, LLC, Fort Lee, NJ (US)

(21) Appl. No.: 17/009,953

(22) Filed: Sep. 2, 2020

Related U.S. Application Data
(63) Continuation-in-part of application No. 16/558,188, filed on Sep. 2, 2019.

(51) Int. Cl. G16H 20/13; G16H 10/60; G16H 40/67; G16H 40/63; A61J 7/04

(52) U.S. Cl. CPC G16H 20/13; G16H 10/60; G16H 40/67; A61J 2200/74; A61J 7/0418; A61J 2200/30; A61J 2205/00; G16H 40/63

(57) ABSTRACT

A method of dispensing medication including: filling a medication container with one or more medication dose, using one or more actuator of a medication dispenser device, according to a prescription for a specific patient, where the prescription is stored in a patient record for the specific patient; verifying delivery of the medication container to a vicinity of the specific patient and/or an environment of the specific patient, where the verifying includes generating a delivery verification signal; verifying use of the one or more medication dose, where the verifying includes generating a use verification signal; and where one or both of the use verification signal and the delivery verification signal are generated using and/or interacting with the medication container.

### RELATED APPLICATION(S)

This application is a Continuation-In-Part (CIP) of U.S. patent application Ser. No. 16/558,188 filed on Sep. 2, 2019, the contents of which are incorporated by reference as if fully set forth herein in their entirety.

### FIELD AND BACKGROUND OF THE INVENTION

The present invention, in some embodiments thereof, relates to medication dispensing and, more particularly, but not exclusively, to a smart medication container for medication dispensing.

### SUMMARY OF THE INVENTION

Following are examples of some embodiments of the invention. Features of one example may be combined with features of one or more other examples, unless expressly prohibited and form additional examples of some embodiments of the invention.

Example 1. A method of dispensing medication comprising:

filling a medication container with one or more medication dose, using one or more actuator of a medication dispenser device, according to a prescription for a specific patient, where said prescription is stored in a patient record for said specific patient;

verifying delivery of said medication container to a vicinity of said specific patient and/or an environment of said specific patient, where said verifying includes generating a delivery verification signal;

verifying use of said one or more medication dose, where said verifying includes generating a use verification signal; and

where one or both of said use verification signal and said delivery verification signal are generated using and/or interacting with said medication container.

Example 2. The method according to Example 1, comprising updating said patient record, using said delivery verification signal, and said use verification signal.

Example 3. The method according to Example 2, comprising evaluating a patient compliance, for a time period, by comparing said use verification signal and said prescription;

wherein said updating comprises updating said patient record using said patient compliance.

Example 4. The method according to Example 3, wherein said one or more medication dose includes medication, according to said prescription, for 1-31 time periods.

Example 5. The method according to Example 3, wherein said one or more medication dose includes medication, according to said prescription, for 1-7 time periods.

Example 6. The method according to Example 3, wherein said filling of said medication container is with a plurality of medication doses, which medication doses fulfil, for said time period, said prescription.

Example 7. The method according to Example 1, comprising evaluating one or more of said delivery verification signal and said use verification signal; and

performing an action, based on said evaluating.

Example 8. The method according to Example 7, wherein said evaluating comprises identifying if a medication dose has been missed; and

wherein said performing comprises one or more of:
initiating an alarm;
adjusting said prescription.

Example 9. The method according to Example 1, wherein said verifying use comprises verifying opening of said medication container.

Example 10. The method according to Example 1, wherein said verifying use comprises verifying removal of medication from said medication container.

Example 11. The method according to Example 1, wherein said verifying use comprises evaluating sensor data.

Example 12. The method according to Example 1, comprising generating a container opening signal, upon opening of said medication container.

Example 13. The method according to Example 12, wherein said verifying use comprises initiating sensing, based on said container opening signal; and

evaluating said sensing.

Example 14. The method according to Example 12, wherein said verifying use comprises sensing; and

evaluating said sensing using timing of said opening signal.

Example 15. The method according to Example 13, wherein said sensing comprises collecting images of said patient.

Example 16. The method according to Example 13, wherein said sensing comprises collecting movement measurements of said patient.

Example 17. The method according to Example 1, wherein said verifying use comprises receiving and evaluating an input at a user interface.

Example 18. The method according to Example 1, wherein said verifying delivery comprises sensing and evaluating said sensing.

Example 19. The method according to Example 1, wherein said verifying delivery comprises receiving and evaluating an input at a user interface.

Example 20. The method according to Example 1, wherein said patient record is hosted by and/or accessed by said medication dispenser.

Example 21. The method according to Example 1, wherein said verifying delivery of said medication container to said specific patient comprises matching a medication container identifier to a patient identifier, using identifier records stored by said medication dispenser.

Example 22. The method according to Example 1, wherein said verifying delivery includes evaluating GPS tracking signal.

Example 23. A medication dispensing system comprising:

a medication container comprising at least one medication compartment and a medication container identifier;

a medication dispenser comprising:
a memory storing a medication prescription for a specific patient registered with said medication container identifier; and
one or more actuator configured to dispense medication, according to said medication prescription, into said at least one medication compartment;

one or more sensor configured to provide a feedback signal associated with one or more of: delivery of said medication container and use of said medication; and

circuitry to evaluate said feedback signal.

Example 24. The medication dispensing system according to Example 23, wherein said circuitry is configured to generate a control signal instructing an action, based on the evaluation.

Example 25. The medication dispensing system according to Example 23, wherein said medication container comprises one or more sensor configured to sense opening of said medication container.

Example 26. The medication dispensing system according to Example 23, wherein said medication container comprises one or more sensor configured to sense removal of medication from said medication container.

Example 27. The medication dispensing system according to Example 26, wherein said one or more sensor comprises a weight sensor.

Example 28. The medication dispensing system according to Example 23, wherein said medication container comprises more than one compartment, each compartment configured to hold at least one dose of medication.

Example 29. The medication dispensing system according to Example 23, comprising one or more sensor configured to sense use of medication.

Example 30. The medication dispensing system according to Example 29, wherein said one or more sensor comprises one or more of:

a camera configured to collect images of said patient; and

one or more patient movement sensor.

Example 31. A method of medication dispensing to a patient comprising:

receiving a medication container opening signal;

upon said receiving, one or more of:
initiating sensing with one or patient environment sensor; and
time marking one or more patient environment sensor signal;

verifying medication use using said one or more sensor signal;

recording said verifying in patient medical records.

Example 32. The method according to Example 31, wherein said upon said receiving comprises supervising said patient.

Example 33. The method according to Example 32, wherein said supervising comprises displaying sensor signals at a user interface.

Example 34. The method according to Example 33, wherein said sensor signals are images of said patient.

Example 35. The method according to Example 32, wherein said verifying medication comprises receiving an input at a user interface.

Example 36. A medication dispenser comprising:

a memory storing a medication prescription for a specific patient registered with a medication container identifier;

one or more actuator configured to dispense medication, according to said medication prescription, into a medication container;

circuitry configured to evaluate one or more feedback signal to verify one or more of: delivery and use of said medication and, based on verification, to evaluate compliance of said specific patient with said medication prescription.

Example 37. A medication container comprising:

at least one closeable compartment configured to store at least one dose of medication;

at least one medication container identifier;

one or more element configured to be used to generate one or both of:
a delivery verification feedback signal, upon delivery of said medication container to a patient vicinity and/or environment; and
a use verification feedback signal, upon use of medication held within said at least one closeable compartment.

Example 38. The medication container of Example 37, comprising a memory configured to store one or more of patient information and medication information.

Example 39. The medication container of Example 37, comprising one or more position tracker.

Example 40. The medication container of Example 37, comprising one or more sensor configured to sense an indication of use of said at least one dose of medication.

Example 41. The medication container of Example 40, wherein said one or more sensor includes a sensor configured to sense opening of said at least one closeable compartment.

Example 42. The medication container of Example 41, wherein said sensor configured to sense opening of said at least one closeable compartment includes an impedance sensor.

Example 43. The medication container of Example 40, wherein said one or more sensor includes a sensor configured to sense removal of medication from said at least one closeable compartment.

Example 44. The medication container of Example 43, wherein said a sensor configured to sense removal of medication from said at least one closeable compartment is a weight sensor.

Example 45. The medication container of Example 37, comprising a disposable housing and a reusable electronics unit.

Example 46. The medication container of Example 37, comprising a plurality of closeable compartments.

Example 47. The medication container of Example 37, wherein said medication container identifier is used to generate said delivery verification feedback signal.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as collecting dental measurements, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified schematic block diagram of a medication dispensing system, according to some embodiments of the invention;

FIG. 2A is a simplified schematic of a medication dispensing system 200, according to some embodiments of the invention;

FIGS. 2B-C are simplified schematics of a container base, according to some embodiments of the invention;

FIG. 3 is a simplified schematic block diagram of a medication dispensing system, according to some embodiments of the invention;

FIG. 4 is a flow chart of a method of medication dispensing, according to some embodiments of the invention;

FIG. 5 is a method of medication dispensing, according to some embodiments of the invention;

FIGS. 6A-B are a flow chart of a detailed method of medication dispensing, according to some embodiments of the invention;

FIG. 7 illustrates two dispensing schedules, according to some embodiments of the invention;

FIGS. 8A-8D are simplified schematics illustrating exemplary use of an medication container, according to some embodiments of the invention;

FIG. 9 is a simplified schematic of a medication container, according to some embodiments of the invention;

FIG. 10 is a simplified schematic of a medication container, according to some embodiments of the invention;

FIG. 11 is a simplified schematic of a medication container, according to some embodiments of the invention;

FIG. 12 is a simplified schematic of a medication container, according to some embodiments of the invention;

FIGS. 13A-C are simplified schematics of a medication container, according to some embodiments of the invention;

FIG. 14 is simplified schematic of a medication container, according to some embodiments of the invention;

FIGS. 15A-B are simplified schematics of a multi-compartment medication container, according to some embodiments of the invention;

FIG. 16 is a simplified schematic of a multi-compartment medication container, according to some embodiments of the invention; and

FIG. 17 is a simplified schematic of a multi-compartment medication container, according to some embodiments of the invention.

### DESCRIPTION OF SPECIFIC EMBODIMENTS OF THE INVENTION

The present invention, in some embodiments thereof, relates to medication dispensing and, more particularly, but not exclusively, to a smart medication container for medication dispensing.

### Overview

A broad aspect of some embodiments of the invention relates to improving reliability of medication dispensing where reliability is improved by verifying delivery to and/or use (e.g. consumption) of medication by a patient. In some embodiments, timing of delivery and/or use (e.g. consumption) is verified. In some embodiments, verifying includes generating a feedback signal which is received by a medication dispenser and/or is incorporated into patient records. Potentially, feedback (e.g. to a medication dispenser) increases reliability of medicating and/or enables tracking of compliance.

An aspect of some embodiments of the invention relates to one or more feedback signal being generated by interaction with and/or using a medication container in which medication is delivered to a patient. In some embodiments, the medication container itself generates one or more feedback signal. Alternatively or additionally interaction with the medication container is used to generate a feedback signal. For example, in some embodiments, the medication container includes one or more passive identifier e.g. an RFID and interaction with this identifier (e.g. using an RFID reader) is used to generate a feedback signal.

In some embodiments, generation of the feedback signal includes receiving a sensor signal, evaluating the signal, and generating a feedback signal based on the evaluating.

In some embodiments, feedback signal/s are generated by a different device/s e.g. within the patient environment e.g. a delivery vehicle, e.g. a medication container docking base. In an embodiment where the medication dispenser itself generates a feedback signal, optionally, additional feedback signal/s are generated by different device/s e.g. within the patient environment.

In some embodiments, feedback signal/s are used to control the medication container e.g. opening and/or unlocking e.g. instructions at a user interface. In some embodiments, the feedback signal is used to adjust the patient prescription, potentially enabling reduction in medication dispensing and/or number of deliveries required in medicating the patient.

In some embodiments, verification is of delivery of an individual prescription of medication to a particular patient. Additionally or alternatively, verification includes verifying use (e.g. consumption) of medication by the patient. Additionally or alternatively, in some embodiments, verifying includes verifying correct supervision for dispensing of medication, supervision being physical and/or remote.

In some embodiments, verifying enables decoupling of delivery of medication to a patient and administering of the medication to the patient. Where, in some embodiments, delivery and administering are at different times and/or are performed by different people. Where, in some embodiments, administering is by a first caregiver physically present and remotely supervised by a second caregiver. A potential advantage being reduction of resources required for delivery and/or dispensing of the medication.

In some embodiments, a locked and/or closed medication container is delivered to a patient. Where, in some embodiments, one or more compartment of a multi-compartment container is unlocked. In some embodiments, compartments have individual locking. In some embodiments, upon one or more type of verification, the medication container unlocks and/or opens (or one or more compartments of a multi-compartment medication container unlock and/or open).

In some embodiments, only an allowed caregiver is able to unlock the medication container. For example, the medication container unlocks only when presented with allowed credentials.

An aspect of some embodiments of the invention relates to evaluating an individual patient's compliance with their medication prescription by verifying delivery of a medication container and/or use of medication within the medication container e.g. using feedback received. Where, in some embodiments, compliance is evaluated for a time period. Where, in some embodiments, the time period is for a single dose of medication (where, dose is defined, in some embodiments, as medication which is to be taken during a time period e.g. together, and, in some embodiments, includes one or more unit (e.g. pill) of medication and/or more than one medication type).

In some embodiments, the medication container includes more than one dose of medication and, in some embodiments, compliance evaluation is performed for each dose, e.g. the time period is no more than that separating doses and/or compliance evaluation is evaluated according to dosing timing data e.g. of the patient's prescription. In some embodiments, compliance is evaluated for more than one dose, but for a small number of doses, for example, 1-20, 1-10, 1-7, or lower or higher or intermediate numbers of doses. In some embodiments, compliance is evaluated at short time intervals, for example, hourly, or daily, or weekly, or at intervals of 2-72 hours, or 2-24 hours, or 2-12 hours, or lower or higher or intermediate intervals.

In some embodiments, verification includes verification that the correct container and/or medication has been delivered to the correct patient. In some embodiments, verification includes verification of timing for medication delivery and/or administration. Where, in some embodiments, timing is part of a prescription.

In some embodiments, verification of delivery includes matching a patient and/or patient space identifier with a medication container identifier. Where the identifiers, in some embodiments were registered by the medication container and/or prior to dispensing and/or delivery of the medication container. In some embodiments, delivery verification includes evaluating sensor data, for example regarding location and/or proximity of the medication container to the patient and/or to a patient space and/or environment (e.g. room). In some embodiments, verification of delivery includes evaluating connection data between the medication container and one or more other device. For example, in some embodiments, verification includes registering disconnection of the medication container from a delivery vehicle and/or connection of the medication container to a delivery base e.g. within the patient space.

In some embodiments, verification of delivery includes evaluating a medication container location e.g. as measured by one or more tracking device. For example, in some embodiments, the medication container and/or a delivery vehicle include position tracker/s.

In some embodiments, verification of medication use includes one or more of verifying that the medication container was opened (e.g. using sensor data), verifying that the medication was removed from the medication container, and evaluating sensing of the patient. Where patient sensing includes in some embodiments, collecting images of the patient which are then analyzed automatically and/or by a supervisor to identify the patient and/or medication use (e.g. consumption) by the patient. Alternatively or additionally, in some embodiments, patient sensing includes patient movement measurements e.g. accelerometer movements of the patient/s body portions. For example, in some embodiments, a patient wrist band includes a motion sensor, and motion sensor measurements are evaluated to verify if medication was consumed or not.

In some embodiments, verification data and/or other data collected by the medication container is received by the medication dispenser. In some embodiments, the verification data is received in real time. Alternatively or additionally, in some embodiments, the verification data is received (e.g. transferred from the medication container to the medication dispenser) when the medication container is returned to the medication dispenser.

In some embodiments, the data then is incorporated into patient records. For example, verification data for a specific medication container and specific patient being stored in the specific patient's medical record.

In some embodiments, a patient's medical record includes a prescription for medication the patient is to receive. For example, the prescription including timing and dosage for each medication. In some embodiments, patient medication record includes other patient information e.g. sex, age, medical history. In some embodiments, one or more part of the patient medical record is stored by a medication dispenser memory and/or care facility electronic records and/or hospital electronic records and/or personal patient electronic records. In some embodiments, a medication dispenser receives patient record/s from care facility electronic records and/or hospital electronic records and/or personal patient electronic records.

In some embodiments, verification data is analyzed, and upon the analysis one or more action is taken. For example, in some embodiments, the patient prescription is adjusted based on the collected verification data. For example, in some embodiments, patient medical record/s are updated with verification/s. In some embodiments, the updated record/s are analyzed and further action/s are performed, based on the analysis. For example, if it is determined that a self-administering patient is not successfully self-administering medication, a patient status is changed to “requiring assistance”. For example, if it is determined that a patient missed one or more dose, procedure with respect to the next medication dose is changed. For example, instructions are issued to administer a subsequent dose at an earlier time and/or to substitute a different medication. Alternatively or additionally, in some embodiments, identification of a missed dose is used to adjust dispensing at the medication dispenser. For example, dispensing and/or delivery of a subsequent medication container is delayed by a time associated with a dose time period. Alternatively or additionally, in some embodiments, identification of a missed dose generates an alert that the patient requires assistance in medication use (e.g. consumption) and/or monitoring. For example, if it is determined that a compartment and/or sub-container has not been opened, medication therein is considered as “not dispensed” enabling the medication, for example, to be used at a different time and/or transferred (e.g. back to the medication dispenser).

Alternatively or additionally, in some embodiments, other data is analyzed, and upon the analysis one or more action is taken. For example, in some embodiments, other data includes patient data, for example, food consumption, patient and/or caregiver inputted data. Where an exemplary action includes adjusting the patient prescription (e.g. changing medication time based on food consumption).

In some embodiments, supervision is verified when a caregiver and/or professional is present, directly supervising. In some embodiments, supervision is remote e.g. using sensor signal/s (e.g. visual from a camera sensor). Where, for example, a caregiver and/or professional examines the sensor signal data and then verifies. In some embodiments, remove supervision is in real time. In some embodiments, remote supervision is at a time period after medicating. In some embodiments, sensor signal/s are saved within a patient's medical record.

In some embodiments, upon unlocking of the container, medication is administered to the patient. In some embodiments, the patient self-administers medication. In some embodiments, administration is performed by a caregiver or the patient is assisted in administration by a caregiver.

In some embodiments, one or more part of verification is triggered by the medication container. For example, in some embodiments, physical delivery of the medication triggers verification that the correct container and/or medication has been delivered to correct patient. For example, in some embodiments, opening of the container triggers verification protocol/s e.g. triggers a process of verifying supervision and/or use (e.g. consumption) of medication.

In some embodiments, one or more part of the delivery, dispensing, and medication administration process is monitored by (and/or monitoring is triggered by) the medication container where, in some embodiments, medication container monitoring data is collected and is incorporated into patient medical records.

In some embodiments, a multi-compartment container is delivered. In some embodiments, different compartments contain doses to be taken by a patient at different times. In some embodiments, different compartments contain medication for different patients.

An aspect of some embodiments of the invention relates to a medication dispenser providing all of a patient's medication requirements, according to the patient's individual prescription. Where, in some embodiments, the medication dispenser prepares medication containers filled with medication dose/s for the specific patient, for their timely use (e.g. consumption) by the patient. In some embodiments, the medication dispenser dispenses medication (e.g. into container/s) periodically which is then delivered to the patient periodically e.g. once dispensed the medication is delivered to the patient. In some embodiments, delivery and/or use of the medication is verified and this verification is used to evaluate patient compliance with the patient's prescription. In some embodiments, the verification and/or compliance evaluation is used to perform an action e.g. to change the prescription and/or generate an alert.

An aspect of some embodiments of the invention relates to a medication container (and/or sub-container) where the container holds a single dose of medication. Where the single dose is defined as medication to be used (e.g. consumed) at a specific time period e.g. the single dose, in some embodiments, include more than one type of medication. Potentially, providing a patient with a container which holds medication to be taken at one time simplifies medication consumption for the patient and/or caregiver potentially improving patient compliance with a prescription. In some embodiments, a medication container holds more than one dose (medication to be taken at a specific time period), but where the doses are separated into different compartments and/or sub-containers. Potentially, such a container maintains advantages of a single dose container but allows more than one dose (medication to be taken at a specific time period) to be delivered to the patient in a single delivery.

A broad aspect of some embodiments of the invention relates to dispensing medication for one or more individual patient into a medication container, where the container is selected based on the prescription (e.g. medication and medication timing) for the one or more patient. In some embodiments, the container is selected from a plurality of different container types. In some embodiments, container/s include more than one compartment for medication. In some embodiments, a container with a plurality of compartments includes individually lockable compartments.

In some embodiments, a container is selected by constructing, at least a portion of the container, based on the prescription e.g. an envelope container is formed by folding.

In some embodiments, a type of container may be filled in more than one way. For example, in some embodiments, a multi-compartment container holds, in each compartment, medication for an individual patient, for different dosing times. For example, in some embodiments, a multi-compartment container holds medication for different individuals in different compartments.

Potentially, being able to dispense medication from storage receptacles (e.g. using a dispenser device) where more than one patient and/or more than one dosing session is dispensed at one time enables increased efficiency in dispensing. For example, in some embodiments, a dispenser device serves a particular number of patients. For example, dispensing multiple doses of medication for patients at the same time opposed to separately dispensing for each patient for each time period potentially reduces dispenser device dispensing time e.g. enabling the device to serve a larger number of patients.

Throughout this document the terms “fill”, “filling”, “filled” when describing a medication container should be taken to mean that medication (e.g. a plurality of doses of medication) has been placed into the medication container, and not necessarily that the medication container's inner volume is entirely filled with medication, and not necessarily that every compartment of a multi-compartment medication container holds medication.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary System

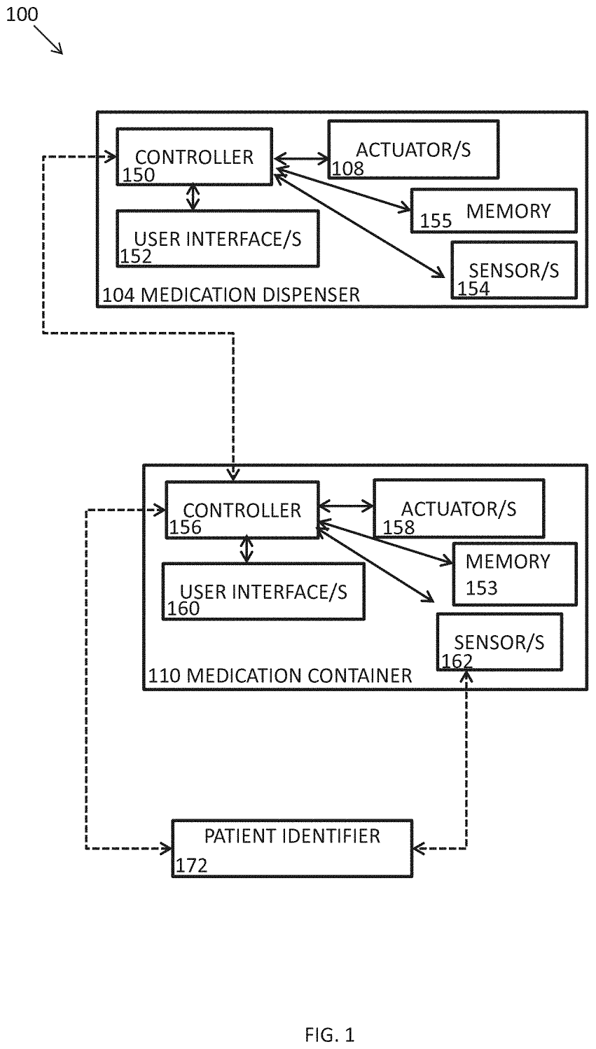

FIG. 1 is a simplified schematic block diagram of a medication dispensing system 100, according to some embodiments of the invention.

In some embodiments, system 100 includes a medication dispenser 104 and a medication container 110.

In some embodiments, medication dispenser 104 is configured to place medication into medication container 110. Where, in some embodiments, medication dispenser actuator/s 108, receiving control signals generated by a controller 150, move medication from medication dispenser 104 to medication container 110.

In some embodiments, medication container 110 is then transferred to a patient. In some embodiments, medication container 110 provides feedback to medication dispenser 104. For example, data regarding whether medication container 110 has been delivered to the correct patient. For example, regarding whether compartment/s of medication container 110 have been opened. For example, regarding whether medication has been removed from medication container 110. For example, regarding whether medication from medication container 110 has been used (e.g. consumed) by a patient. For example, location of medication container 110 (e.g. as provided by one or more position tracker e.g. GPS tracker).

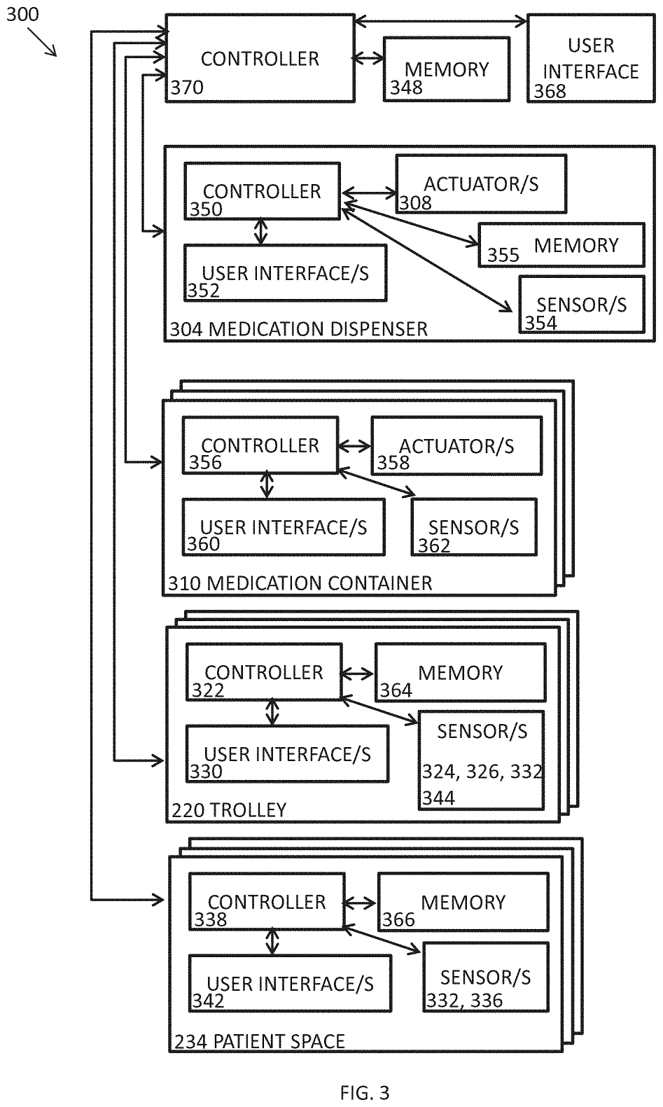

In some embodiments, medication container 110 communicates directly with medication dispenser 104 e.g. controller 156 of the medication container communicating with controller 150 of the medication dispenser. For example, wirelessly. For example, through a direct connection e.g. when medication container is returned to the medication dispenser e.g. after use. In some embodiments, medication container 110 communicates through an intermediary, e.g. one or more of intermediary controllers 370, 322, 338 e.g. as illustrated in FIG. 3.

In some embodiments, medication container 110 includes one or more actuator 158. In some embodiments, actuator/s 158 are configured to one or more of, open, close, unlock, lock the medication container 110 to provide (or deny) access to medication within the container. In some embodiments, actuator/s are controlled by controller 156 receiving control signal/s sent by controller 156.

In some embodiments, medication container 110 includes a memory 153.

In some embodiments, medication container 110 includes one or more user interface 160. In some embodiments, user interface 160 includes one or more touch input button and/or a visual display (e.g. a touch screen) and/or an audio speaker.

In some embodiments, user interface 160 is configured to communicate information to a user (e.g. patient and/or caregiver):

For example, whether container compartment/s have been opened.

For example, whether environmental conditions of the container have potentially affected the medication within the container e.g. exposure to extreme/s of temperature and/or humidity. Where, for example, environmental condition sensor data (e.g. from container sensor/s) is used to provide the information to be communicated to the user.

For example, information regarding the medication container's designated patient e.g. patient age group, e.g. chronic pathologies of the patient, e.g. handicap status, e.g. whether the patient requires assistance for medication administration.

For example, information regarding the content/s of the medication container e.g. type of medication e.g. prescription details e.g. including medication administration instruction/s.

For example, in some embodiments, at least a portion of the container has a color which is configured to be changed. Color changes, for example, providing information to a user e.g. designated patient age group designated by color.

In some embodiments, user interface 160 is configured to receive inputs from a user. For example, includes one or more button and/or switch. For example, alternatively or additionally, includes one or more optical sensor e.g. for face and/or fingerprint recognition. For example, alternatively or additionally, includes a touch screen.

In some embodiments, medication container 110 includes one or more sensor 162. In some embodiments, sensor/s 162 sense presence and/or absence of medication within medication container 110 e.g. a weight sensor.

In some embodiments, sensor/s 162 sense opening of compartment/s of medication container 110. For example, in some embodiments, sensor/s 162 include one or more impedance sensor configured to sense opening of the medication container. For example, in some embodiments, sensor/s 162 include one or more strain sensor e.g. configured to sense opening of the medication container (e.g. the strain sensor mounted on a hinge of a medication container compartment cover).

In some embodiments, sensor/s sense the environment of the medication container and/or of the patient e.g. temperature and/or humidity.

In some embodiments, medication container includes one or more tracking device configured to provide a signal associated with a position of the medication container in space and/or with respect to a patient and/or patient identifier.

In some embodiments, system 100 includes a patient identifier 172. In some embodiments, medication container 110 interacts with patient identifier 172 to produce feedback to medication dispenser 104. For example, when verifying that the correct medication container has been delivered to the correct patient. For example, in some embodiments, patient identifier 172 includes an RFID tag and sensor/s 162 include an RFID reader. In some embodiments, system 100 includes fingerprint and/or face recognition capabilities. For example, including hardware (e.g. optical sensor/s) and/or software.

In some embodiments, system 100 includes more than one medication container 110. In some embodiments, system 100 includes more than one medication dispenser 104 where, in some embodiments, a specific medication container communicates with the medication dispenser which filled the specific container. In some embodiments, system 100 includes both a plurality of medication containers 110 and a plurality of medication dispensers 104 and one or more of the plurality of medication containers communicates with more than one (e.g. all) of the medication dispensers.

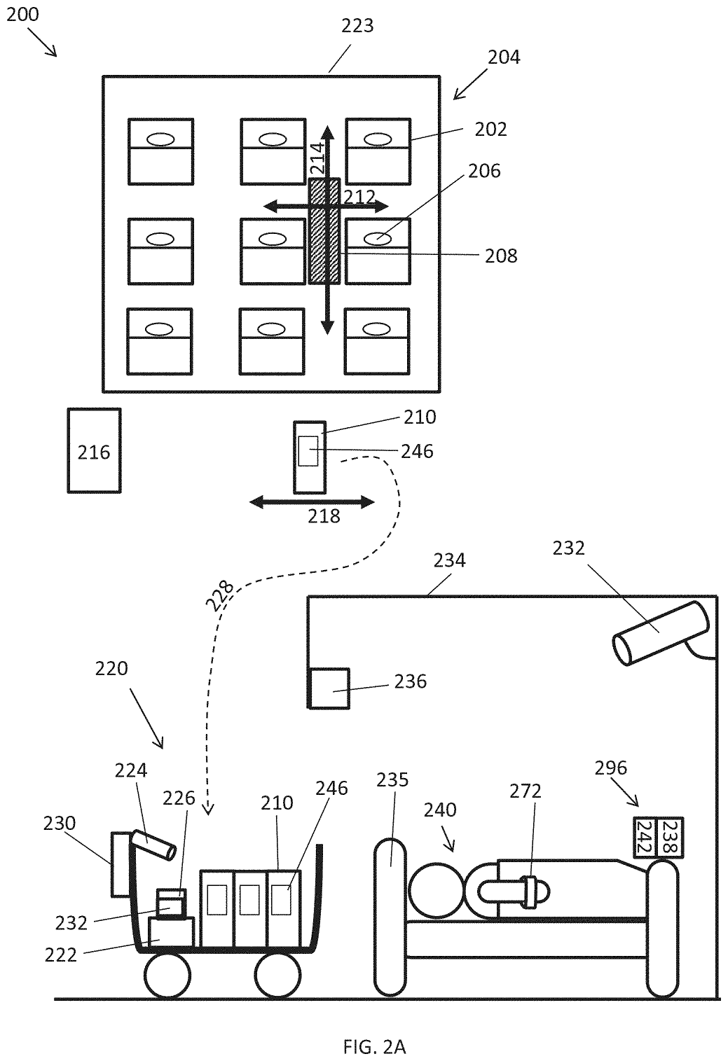

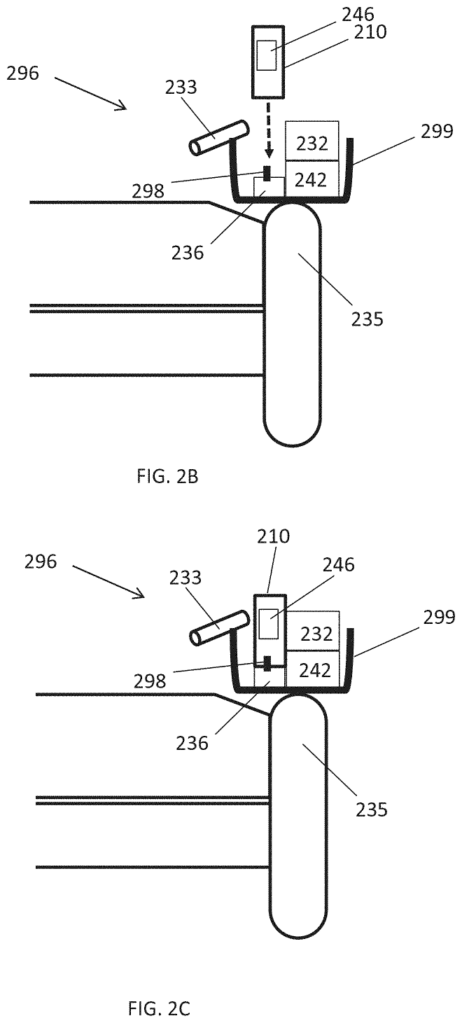

FIG. 2A is a simplified schematic of a medication dispensing system 200, according to some embodiments of the invention.

In some embodiments, system 200 includes a medication dispenser 204. In some embodiments, medication dispenser 204 includes a plurality of medication storage receptacles 202. In some embodiments, receptacles 202 are held in position by a support structure 223.

In some embodiments, medication (e.g. for a particular patient) is transferred from storage receptacle/s 202, into a medication container 210. Where, in some embodiments, medication from more than one storage receptacle 202 (e.g. different medications each stored in a different storage receptacle) are transferred into medication container 210.

In an exemplary embodiment, a probe configured to remove medication from the receptacle/s (e.g. by suction) is moved (e.g. by one or more actuator) between receptacles (movement e.g. as illustrated by arrows 212, 214) to extract medication through receptacle openings 206, and release the medication which, for example, falls under gravity into container 210.

In some embodiments, medication container 210 is filled by removing a single unit of medication (e.g. a single pill) from a receptacle (e.g. using the probe) and placing it within the medication container before, optionally, in some embodiments, removing more unit/s from receptacle/s.

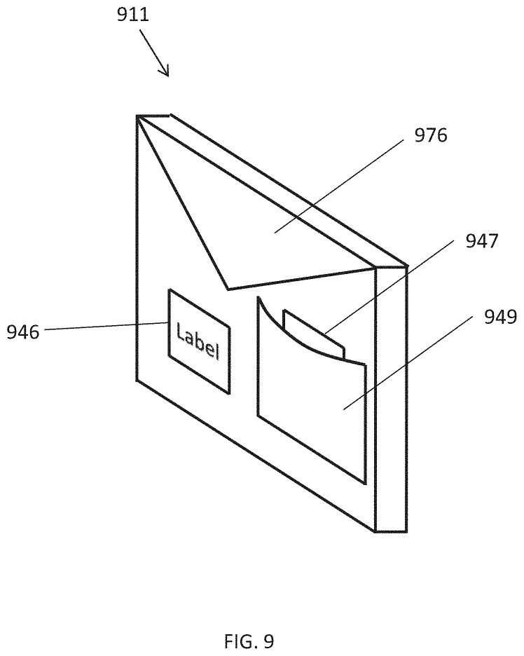

In some embodiments, medication container 210 is moved as indicated by arrow 218 (e.g. by one or more actuator), the movements following movements of the probe for collection of released medication within medication container 210. In some embodiments, one or more sub-container is filled and then placed in medication container 210 (sub-container e.g. including one or more feature as illustrated and/or described regarding sub-container and/or medication container 911 FIG. 9).

In some embodiments, a sub-container includes edible material, edible parts of the sub-container which also contain medication being consumed by the patient. For example, in some embodiments, a sub-container is an edible (e.g. rice-paper) envelope which holds medication.

In some embodiments, system 200 includes a stock pile of a plurality of containers. In some embodiments, a plurality of containers 216, are loaded to the medication dispenser 204.

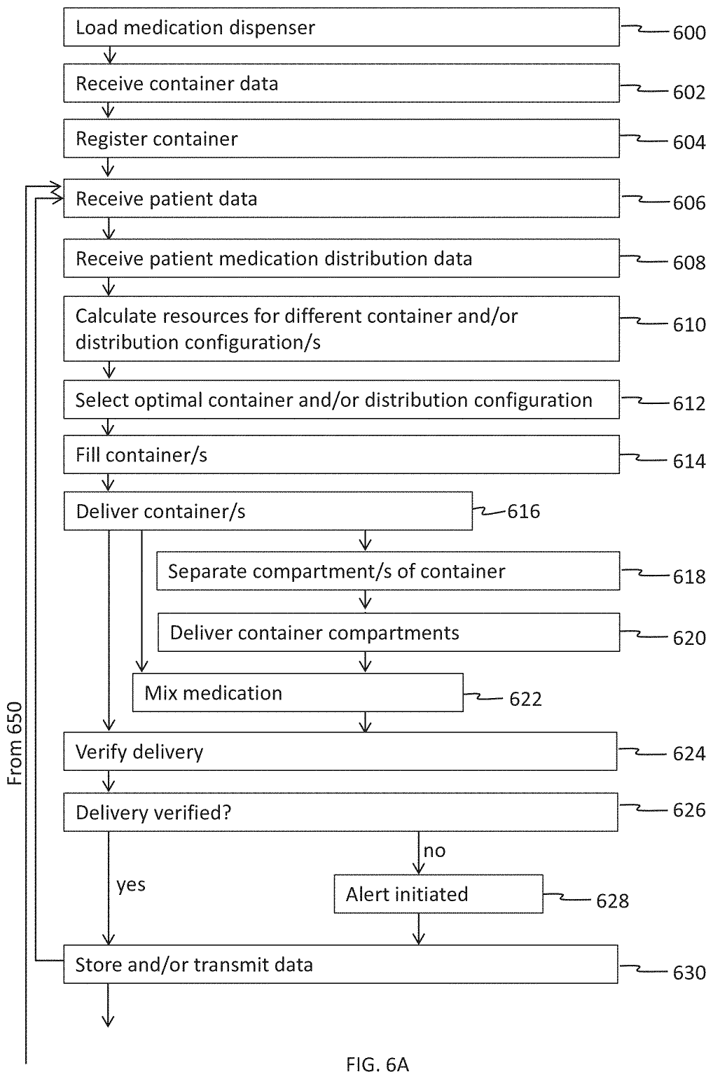

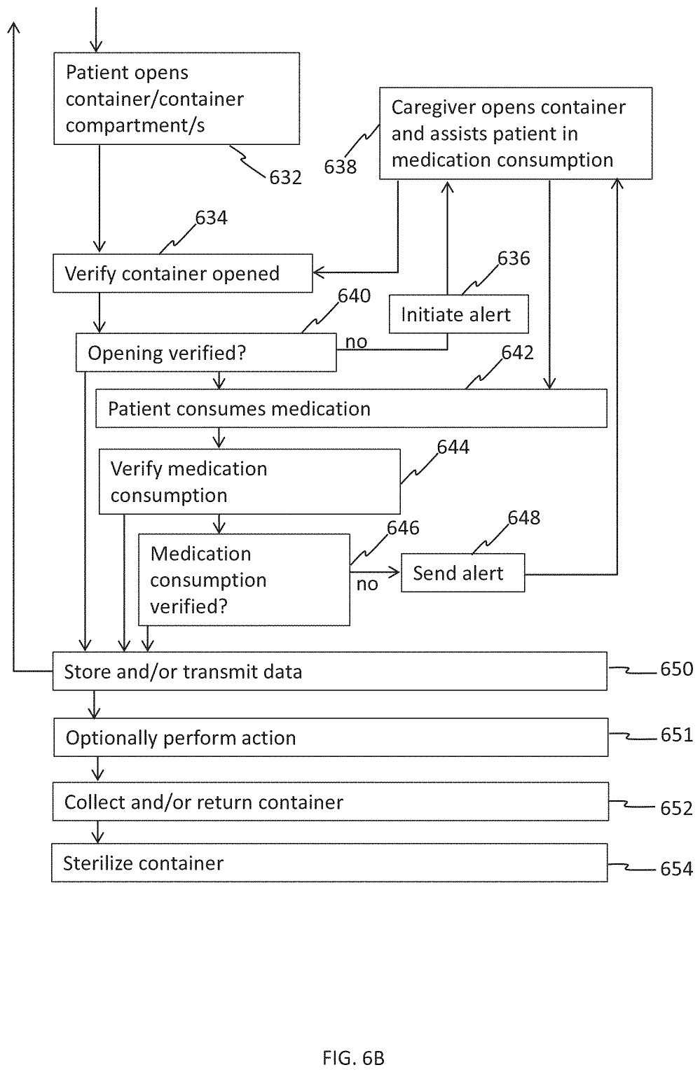

In some embodiments, loading of containers which have been previously used includes transfer of data from the containers to the medication container e.g. as described regarding step 602 FIG. 6A. For example, in some embodiments, loading of containers includes making a data connection between the medication container and the medication dispenser e.g. plugging the container into the medication dispenser or vice versa e.g. scanning the container.

Alternatively or additionally to loading containers, medication dispenser 204, in some embodiments, at least partially constructs containers. For example, using construction materials 216 e.g. paper and/or card from which containers 210 are constructed e.g. by folding and/or cutting and/or connecting (e.g. sticking). In some embodiments, construction material 216 include different sizes of material e.g. different sheet sizes of paper and/or card e.g. for forming different sized containers. In some embodiments, construction material 216 include one or more of fabric, plastic, metal, paper, and cardboard.

In some embodiments, medication container 210 includes a single medication-containing compartment. In some embodiments, medication container 210 includes a plurality of medication-containing compartments.

In some embodiments, one or more part of functionality as illustrated in and/or described regarding medication container 110 in FIG. 1 is external to medication container 210 e.g. not hosted by the medication container itself. For example, one or more part of functionality of medication container 110 being within patient space 234, but not part of the medication container 210. For example, hosted by a delivery vehicle 220. For example, hosted by a medication container base 296. Potentially, simplifying medication container 210 reduces complexity and/or cost of the container e.g. of a single use medication container and/or of a medication container with a limited number of use/s (e.g. 1-100, or 1-10, or lower or higher or intermediate number of uses).

In some embodiments, medication container 210 includes one or more identifier 246. In some embodiments, medication dispenser 204 registers dispensing data for medication container 210 (e.g. medication and/or patient data) with identifier 246. Where registering is, for example, stored in a memory (e.g. medication dispenser memory 355 and/or system memory 348 FIG. 3).

In some embodiments, medication container identifier 246 includes active components of medication container 210 (not illustrated in FIGS. 2A-C). For example, referring back to FIG. 1, medication container 110 identifier is hosted by medication dispenser controller 150 and/or memory 155. In some embodiments, patient and/or medication data (e.g. sent to the container by the medication container) is stored on the container memory 155.

In some embodiments, medication container 210 includes one or more passive identifier 246. In some embodiments, medication container 210 does not include active elements. In some embodiments, identifier 246 includes a label e.g. including patient and/or medication detail/s and/or a machine readable label e.g. barcode, QR code. Alternatively or additionally, in some embodiments, identifier includes a machine readable identifier e.g. an RFID.

In some embodiments, medication dispenser 204 generates and/or attaches identifier 246 to the medication container 210. For example, in some embodiments, medication dispenser 204 prints a label identifier 246. For example, in some embodiments, medication dispenser 204 attaches identifier 246 to medication container.

In some embodiments, containers 210 are transported 228 from dispenser 204 to a patient 240 or patient space 234 (e.g. patient room). For example, in some embodiments, filled containers 210 are placed into a delivery vehicle 220 (e.g. delivery trolley) and transported towards the patient and/or patient space 234.

In some embodiments, delivery vehicle 220 is autonomous e.g. robotically transports containers 210 e.g. from medication dispenser 204 to patient 240 and/or patient space 234.

In some embodiments, delivery vehicle 220 includes one or more passive and/or active identifier. Passive identifier/s including, for example, machine readable label/s and/or RFID/s. Active identifier/s including, for example, a delivery vehicle controller 222 and/or a delivery vehicle memory.

In some embodiments, the delivery vehicle identifier is registered with medication container identifier/s 246 of medication container/s 210 placed within delivery vehicle 220. Where registering is, for example, stored in a memory (e.g. the vehicle delivery memory and/or a medication dispenser memory 355 and/or system memory 348 FIG. 3).

In some embodiments, delivery vehicle 220 includes one or more sensor 224, 226. For example, one or more optical sensor 224 (e.g. camera) and/or one or more weight sensor 226. Where, in some embodiments, sensor/s 224, 226 sense insertion and/or presence and/or removal of medication container/s 210.

In some embodiments, delivery vehicle 220 includes a container identifier unit 232. For example, where container identifier unit 232 sends and/or receives a signal to identify containers 210 within the delivery vehicle 220. For example, in some embodiments, container identifier unit 232 includes an RFID reader e.g. able to read RFID container identifiers 246.

In some embodiments, delivery vehicle 220 includes one or more user interface 230. In some embodiments, user interface 230 displays instruction/s and/or indication/s to a user. For example, instructions indicating which patient to give a specific medication container to. For example, indication/s that a medication container has been verified with a patient. In some embodiments, user interface 230 is configured to receive inputs from user/s e.g. presence and/or absence of a patient e.g. changes to a patient prescription as issued by a physician in proximity to delivery vehicle 220.

In some embodiments, delivery vehicle controller 222 receives sensor data from one or more of sensor/s 224, 226, 232 and/or from user interface 230. In some embodiments, controller 222 saves and/or communicates feedback signal/s to medication dispenser and/or a central controller and/or memory and/or server.

In some embodiments, patient 240 and/or a patient's space 234 (e.g. room) is monitored by one or more sensor 232, 236. For example, an optical sensor 232 (e.g. camera). For example, a sensor 236 sensing presence of trolley 220 and/or medication container 210. In some embodiments, sensor/s 232, 236 monitor patient 240. For example, in some embodiments, camera 232 is configured to collect images of patient 240.

In some embodiments, a medication container 210 is delivered to a patient 240. In some embodiments, patient 240 and/or a patient space 234 (e.g. patient room) include one or more patient identifier 236, 272. In some embodiments, patient identifier/s 236, 272 are passive e.g. include an RFID tag. Additionally, or alternatively, in some embodiments, patient identifier/s 236, 272 are active e.g. including one or more sensor e.g. include an RFID reader or other sensor. In some embodiments, a patient identifier 236 is attached (e.g. to patient clothing e.g. a tag) to patient 240 and/or is worn by patient 240 e.g. is part of a wristband 272 as illustrated in FIG. 2A.

In some embodiments, one or more patient identifier 272 (passive and/or active) is hosted by one or more object, for example, an object which the patient carries or is near to generally. For example, a patient electronic device (e.g. cell phone, tablet, lap top computer). For example, a patient bag, coat, shoe.

In some embodiments, a container base 296 includes a controller 238 and/or user interface 242 is associated with patient 240 and/or a patient space e.g. room 234 and/or bed 235. In some embodiments, one or more patient identifier 272 is hosted by base 296. In some embodiments, instruction/s and/or indication/s to the patient and/or caregiver are issued by user interface 242. In some embodiments, user interface 242 receives patient and/or caregiver inputs.

In some embodiments, one or more element within patient space 234 is hosted by container base 296. Alternatively, container base is a component in system 200, additional to element/s within patient space 234.

In some embodiments, one or more portion of functionality as described regarding medication dispenser 104 FIG. 1 and/or medication dispenser 204 FIG. 2A and/or medication dispenser 304 FIG. 3 is hosted by one or more device. For example, by a system of a plurality of such medication dispensers. For example (alternatively or additionally) by device/s in communication with the medication dispenser/s e.g. personal mobile device/s e.g. remote server/s e.g. cloud server/s.

In some embodiments, medication dispenser 104 FIG. 1 and/or medication dispenser 204 FIG. 2A includes at least one redundant and/or backup hardware/module where said at least one hardware/module can be removed and/or replaced from said pharmaceutical dispensing machine without interrupting the functioning of the machine. For example, the medication dispenser including one or more feature as described and/or illustrated regarding the medication dispenser (termed modular pharmaceutical dispensing machine) described and/or illustrated, for example in U.S. Pat. No. 10,614,916 which is herein incorporated in its entirety by reference.

In some embodiments, medication dispenser 104 FIG. 1 and/or medication dispenser 204 FIG. 2A is configured to store a plurality of different individual pharmaceuticals and to prepare, in a unique same container, a plurality of different individual pharmaceuticals, to be taken by a user, where single pills and/or pharmaceuticals are picked and inserted in the unique same container). For example, For example, the medication dispenser including one or more feature as described and/or illustrated regarding the medication dispenser (termed the pharmaceutical dispensing machine) of international patent application IB2020/052052 which is herein incorporated in its entirety by reference.

In some embodiments, medication dispenser 104 FIG. 1 and/or medication dispenser 204 FIG. 2A includes one or more features of (and/or methods are employed regarding) the medication dispenser (termed mobile pharmaceutical dispensing machine), as described and/or illustrated in U.S. Patent Application No. 63/014,145 which is herein incorporated in its entirety by reference.

FIGS. 2B-C are simplified schematics of a container base 296, according to some embodiments of the invention.

In some embodiments, container base 296 includes one or more sensor 236, 233. For example, a sensor 236 configured to sense presence of a medication container 210. In some embodiments, container base includes an optical sensor 233 e.g. a camera which, for example, in some embodiments, is configured to collect images of the patient.

In some embodiments, container base 296 includes a controller and/or user interface. In some embodiments, container base 296 includes a connector 298 which provides one or more of data connection, electrical connection (e.g. for charging a medication container battery), and mechanical connection between container base 296 and medication container 210. In some embodiments, FIG. 2B illustrates medication container 210 before connection with base 296 and FIG. 2C illustrates medication container 210 after connection with base 296.

In some embodiments, container base 296 is configured to be attached to a portion of patient space, e.g. to bed 235. In some embodiments, container base 296 includes a housing 299.

FIG. 3 is a simplified schematic block diagram of a medication dispensing system 300, according to some embodiments of the invention.

In some embodiments, FIG. 3 is a block diagram of the system illustrated in FIG. 2A.

In some embodiments, system 300, includes at least one medication dispenser 304, one or more medication container 310, one or more medication delivery trolley 320 and one or more patient room 334.

Optionally, in some embodiments, system 300, includes one or more central element. For example, one or more of a central controller 370, a central memory 348, and a central user interface 368. In some embodiments, central elements 370, 348, 368 are cloud hosted, or hosted on a server remote to medication dispenser 304. In some embodiments, one or more of central elements 370, 348, 368 are hosted by a server, e.g. a server remote to medication dispenser 304.

In some embodiments, central controller 370 communicates with more than one medication dispenser. Potentially enabling a patient to be dispensed medication from more than one medication dispenser and/or for a patient to transfer between receiving medication a dispenser (e.g. in a first location) to another dispenser (e.g. in a second location).

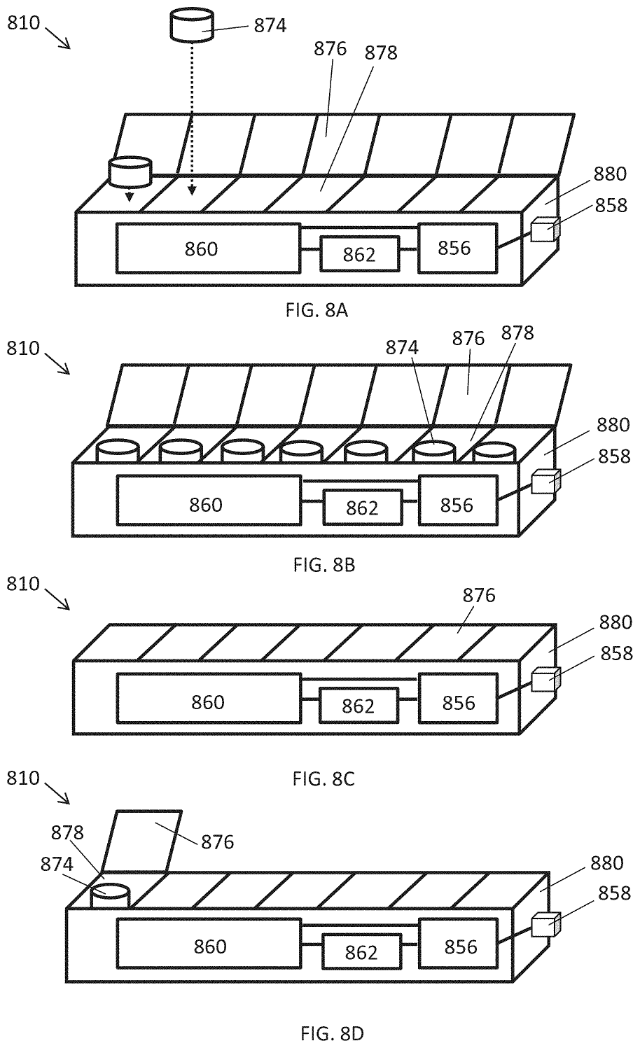

In some embodiments, medication dispenser 304 includes one or more actuator 308 (actuator/s, in some embodiments, including one or more feature as illustrated in and/or described regarding actuators 208, 218 FIG. 2A, and/or actuator/s 858 FIGS. 8A-D). In some embodiments, medication dispenser 304 includes one or more user interface 352. In some embodiments, medication dispenser 304 includes one or more sensor 354.

In some embodiments, medication container 310 includes one or more of a controller 356, one or more actuator 358, one or more sensor 362 and one or more user interface 360.

In some embodiments, delivery vehicle 320 includes one or more of a controller 322, a memory 364, one or more user interface 330, one or more sensor 324, 326, 332, 344.

In some embodiments, patient space 334 includes one or more of a controller 338, a memory 366, one or more sensor 332, 336, and one or more user interface 342.

### Exemplary Methods

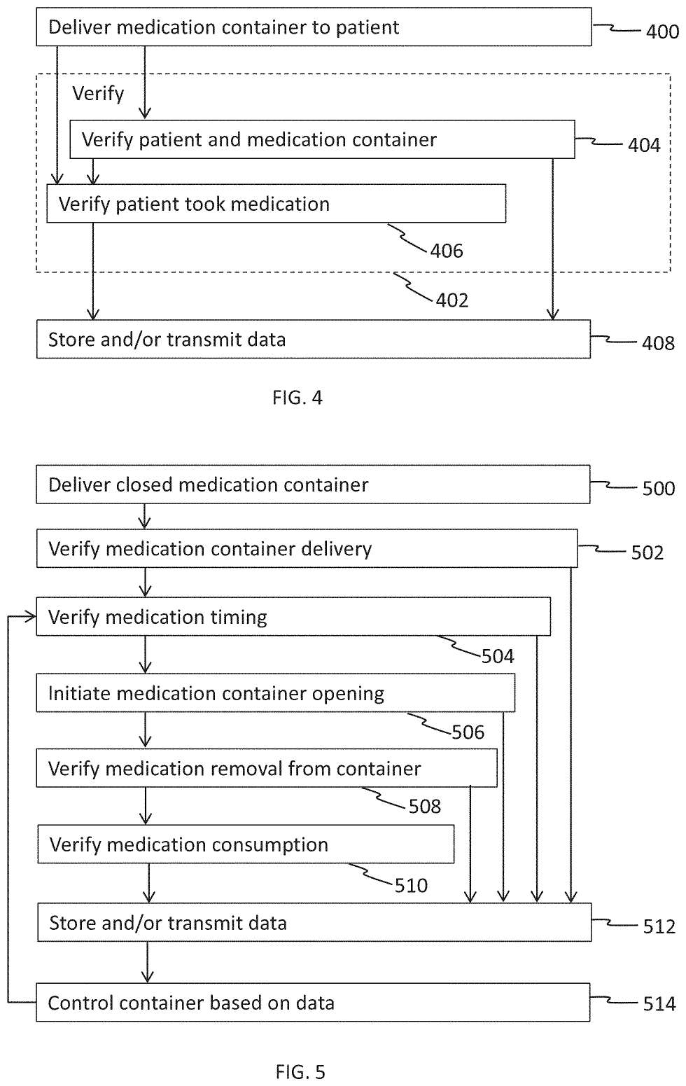

FIG. 4 is a flow chart of a method of medication dispensing, according to some embodiments of the invention.

At 400, in some embodiments, medication is delivered to a patient. For example, a medication container containing medication is delivered to a patient space.

At 402, in some embodiments, verification is performed.

At 404, in some embodiments, verification includes verifying that the particular medication container has been delivered to the correct patient.

At 406, in some embodiments, verification includes verifying that the patient consumed the medication, where verification optionally includes timing of the medication use (e.g. consumption).

At 408, in some embodiments, verification data from step 404 and/or 406 is stored and/or transmitted, for example to a medication dispenser.

FIG. 5 is a method of medication dispensing, according to some embodiments of the invention.

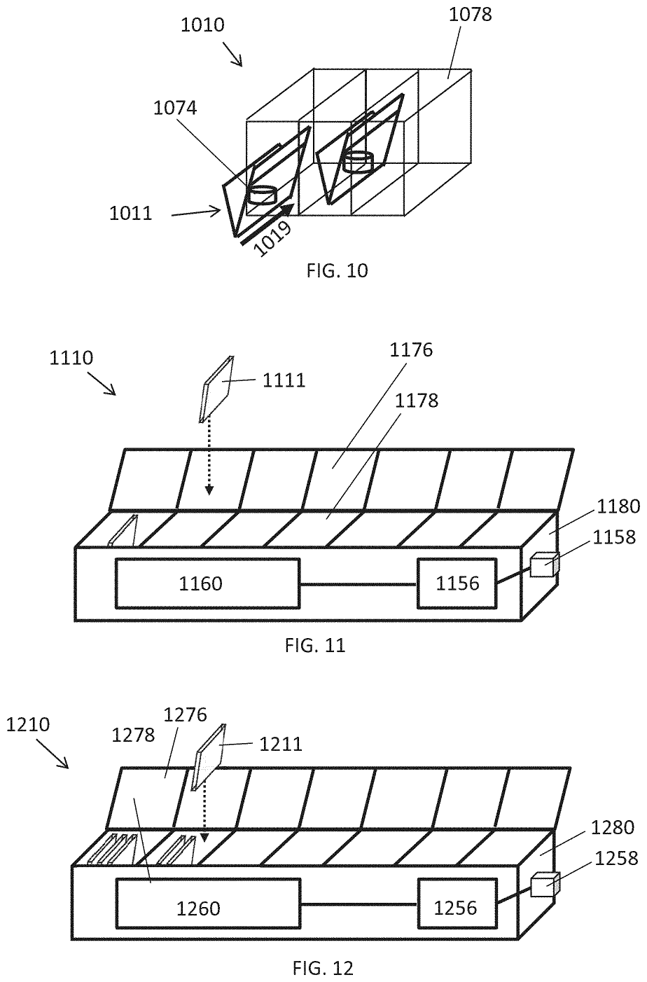

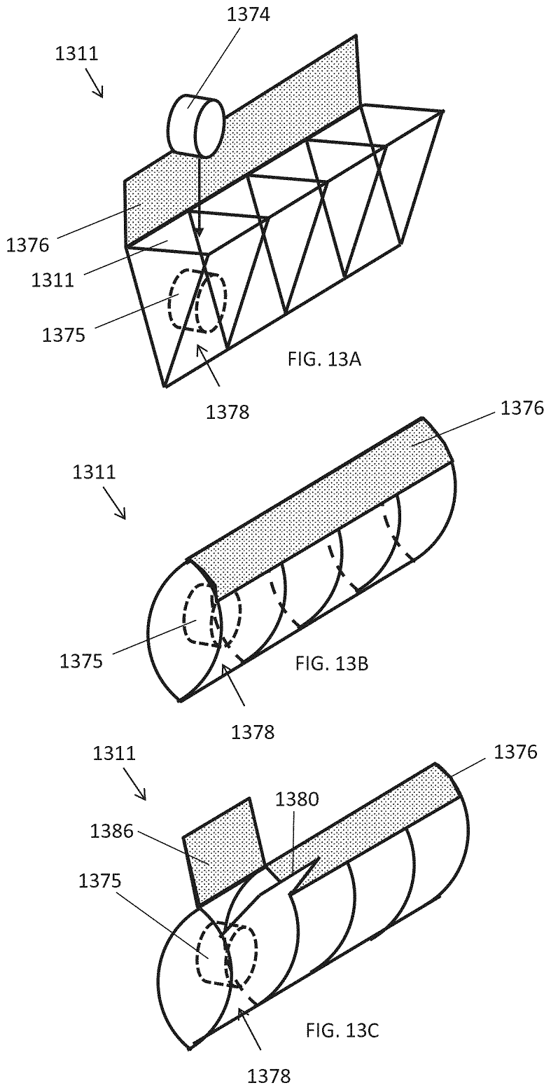

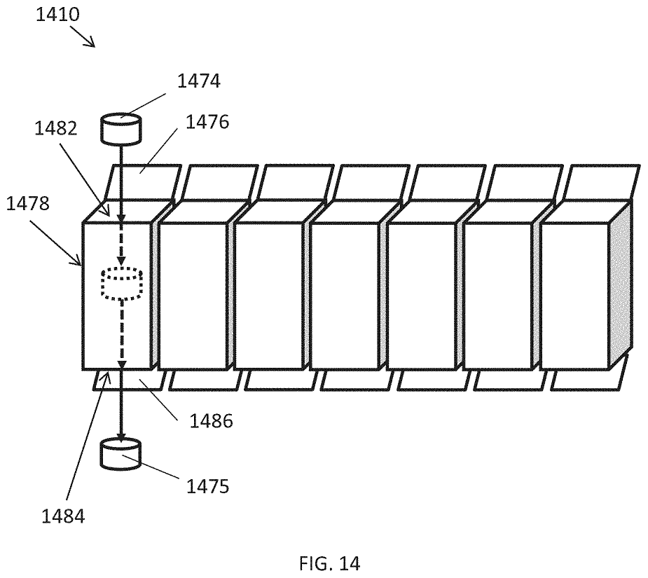

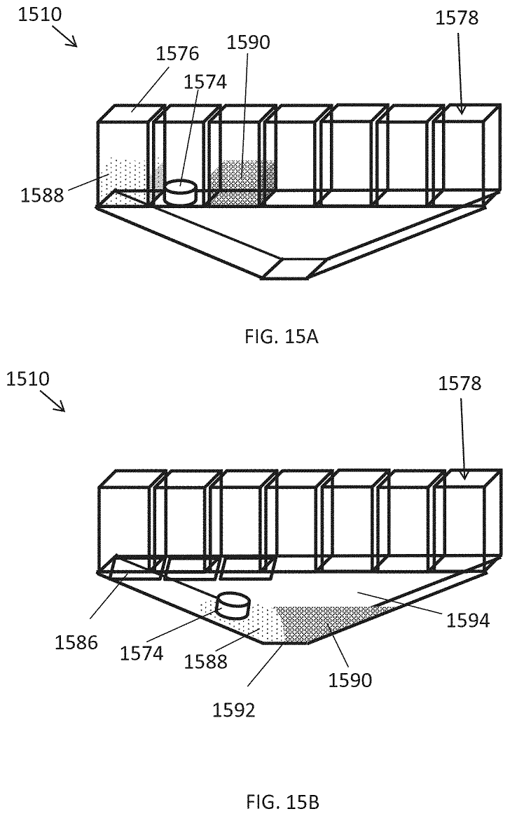

At 500, in some embodiments, a closed medication container (e.g. medication container 110 FIG. 1, 210 FIG. 2A, 310 FIG. 3, 810 FIGS. 8A-D, 911 FIG. 9, 1010 FIG. 10, 1110 FIG. 11, 1210 FIG. 12, 1310 FIGS. 13A-C, 1410 FIG. 14, 1510 FIGS. 15A-B) is delivered. Where delivery, in some embodiments, includes transferring the medication container to a patient environment (e.g. room, ward, in a vicinity of one or more portion of the patient environment e.g. bed, e.g. base). Where transfer, in some embodiments, is from a medication dispenser which filled the medication container with medication.

At 502, in some embodiments, delivery of the medication container is verified.

In some embodiments, verification includes verifying that the medication container has arrived at a patient environment. For example, based on one or more sensor signal.

In some embodiments, verification includes checking that the specific medication container has arrived to a matching patient environment.

Where checking, in some embodiments is that two or more identifiers match e.g. whether a medication container identifier matches a patient identifier. In some embodiments, checking is performed by the medication container itself e.g. by a medication container controller (e.g. controller 156). Alternatively or additionally, in some embodiments, a delivery vehicle controller checks the identifiers and/or signal data is sent to a remote controller (e.g. a central controller and/or a medication dispenser controller).

In some embodiments, verifying includes verifying that the medication container has been removed from a delivery vehicle (e.g. delivery vehicle 220 FIG. 2A). Where verification of removal from a delivery vehicle, in some embodiments, includes evaluating medication container motion sensor data (e.g. accelerometer data) and/or evaluating disconnection of the medication container and/or evaluating data regarding distance of the medication container from the delivery vehicle.

In some embodiments, verifying includes verifying that the medication container has been placed within a medication container base (e.g. base 296 FIGS. 2A-C).

In some embodiments, verification includes checking a GPS location of the medication container and/or delivery vehicle. Where, in some embodiments one or more of the medication container and/or delivery vehicle include a GPS tracker.

At 504, in some embodiments, timing for medication dosing is verified.

At 506, in some embodiments, opening of the medication container is initiated. In some embodiments, the container opens (e.g. one compartment of a multi-compartment container) automatically (e.g. one or more actuator opens the container). In some embodiments, the medication container unlocks automatically, allowing removal of the mediation. In some embodiments, an instruction is issued to a user (e.g. patient or caregiver) to take medication. In some embodiments, the user is instructed what medication to take, for example, opening the container allows access to more than one dose and/or more than one medication type, and the user interface communicates instructions as to what medication the patient should take.

In some embodiments, initiating opening of the medication container is upon successfully verifying at steps 403 and/or 405. Optionally, in some embodiments, appropriate supervision is verified. Where, for example, an individual qualified to supervise medication use (e.g. consumption) and/or administer medication is physically and/or remotely supervising. Potentially, verifying before initiating opening of the container prevents errors in medicating the patient.

In some embodiments, a signal is generated when the medication container is opened. For example, by medication container sensor/s. For example, by sensor/s within the medication container vicinity. In some embodiments, the signal is received and/or stored by the medication dispensing system e.g. in patient records. In some embodiments, the signal is used to verify that the medication container has been opened.

At 508, in some embodiments, medication removal from container is verified. For example, using sensor signal/s e.g. a medication container weight sensor e.g. optical sensor data from which patient and/or medication movements are extracted.

At 510, in some embodiments medication use (e.g. consumption) is verified, for example, from optical sensor data which patient and/or medication movements are extracted.

In some embodiments, sensor data received from a first sensor is used to control and/or process data received from a second sensor. For example, in some embodiments, a sensor is activated and/or sensor data is processed for verifying medication use (e.g. consumption), based on timing of sensor signal/s in verification at step 407 and/or 409.

At 512, in some embodiments, data collected in one or more of steps 403-411 is stored and/or transmitted. For example, where processing involved in verifying occurs remote to where signals used for verifying are generated. Stored, for example, potentially enabling tracking of medication use (e.g. consumption) and/or generation of feedback based on the data e.g. change/s to the medication prescription.

At 514, in some embodiments, operation of the medication container and/or medication dispenser is controlled, based on verification data. For example, in the event that a patient misses a dose, e.g. verification fails, timing at step 405 is adjusted for future doses/s and/or medication to be dispensed in future by the medication dispenser is changed. For example, in some embodiments, sensed patient activities are used to control the medication container. For example, in some embodiments, patient food consumption is monitored (e.g. by monitoring patient position e.g. in a care facility) so that the patient is administered and/or self-administers medication before, with or after eating e.g. according to the patient prescription.

### Exemplary Detailed Method

FIGS. 6A-B are a flow chart of a detailed method of medication dispensing, according to some embodiments of the invention.

In some embodiments, dispensing is by a medication dispenser including one or more feature as described regarding and/or illustrated system 100 FIG. 1, system 200 FIGS. 2A-C and/or system 300 FIG. 3.

At 600, optionally, in some embodiments, the medication dispenser is loaded.

In some embodiments, one or more medication container is loaded to the medication dispenser. In some embodiments, the system is compatible with more than one type of container. In some embodiments, re-usable containers are loaded, where, for example, in some embodiments, used containers are loaded to the system for re-use. In some embodiments, single use containers are loaded into the system.

Alternatively or additionally, in some embodiments, material for construction of containers and/or sub-containers is loaded to the medication dispenser.

At 602, optionally, in some embodiments, the system receives data from one or more medication container.

In some embodiments, containers loaded to the system transfer data to the system. Where, for example, in some embodiments, loading creates a data connection between a container and the system. For example, a hard data connection e.g. pin/s forming a data connection between the container and system and/or a soft data connection e.g. a wireless connection.

In some embodiments, medication container data is transferred wirelessly from the container to the system. In some embodiments, medication container data is transferred to an intermediate space (e.g. to a cloud server, to a memory of an intermediate electronic device). In some embodiments, the data is then transferred to the system.

At 604, in some embodiments, a container is registered, for example, with the medication dispenser.

In some embodiments, registering the container includes detecting a container identifier (using one or more system sensor) and registering the container identifier. In some embodiments, the medication dispenser generates the medication container identifier.

In some embodiments, registering of the medication container is during data transmission between the container and the medication dispenser e.g. as described in step 602.

At 606, in some embodiments, the system receives patient data. For example, for one or more patient, a patient prescription (e.g. what medication the patient requires, at what time and/or at what frequency).

In some embodiments, a plurality of patient prescriptions are received. For example, by a medication dispenser (e.g. medication dispenser 104 FIG. 1, 204 FIG. 2A, 304 FIG. 3). For example, by a controller of a medication container e.g. controller 156 FIG. 1, 356 FIG. 3.

Where, in some embodiments, the prescription is received from a memory, for example, an internal medication dispenser memory (e.g. memory 155 FIG. 1, 355 FIG. 3) and/or from an external memory e.g. memory 348 FIG. 3.

In some embodiments, patient prescription/s are inputted at a user interface e.g. by a prescribing physician e.g. user interface 368 and/or user interface 352 FIGS. 2A-C.

In some embodiments, data is received from a remote source. For example, data stored on an external memory e.g. cloud server. Alternatively or additionally, in some embodiments, data is stored in a system memory (e.g. central memory FIG. 3). In some embodiments, the system receives patient data from one or more medication container e.g. including one or more feature as described regarding step 602.

In some embodiments, patient data associated with prior dispensing of medication is received. For example, data from one or more of steps 630 and 650.

At 608, in some embodiments, the system receives patient medication distribution data. For example, one or more of a patient location, a patient medication distribution point identifier, a patient care organization, a patient care-giver identifier. In some embodiments, data is received from a remote source. For example, data stored on an external memory e.g. cloud server. Alternatively or additionally, in some embodiments, data is stored in a system memory. In some embodiments, the system receives patient medication distribution data from one or more medication container e.g. data transfer as described regarding step 402.

At 610, in some embodiments, resources needed for different medication container configurations are calculated. For example, dispensing resources and/or distribution resources.

Where, a container configuration, in some embodiments, includes a container type for each patient prescription.

In some embodiments, for one or more of the patient prescriptions a medication container is selected, based on the patient prescription. In some embodiments, other patient data is used in selecting medication container/s. For example, patient characteristic/s associated with opening of the medication container (e.g. easy-opening for disabled and/or older patients).

In some embodiments, selection of a container includes selecting a type of container, from a plurality of types of container.

In some embodiments, selection of a container includes selecting a number of doses and/or prescriptions to be held by the container.

In some embodiments, selection of a container includes selecting a number of compartments of the container.

In some embodiments, selection of a container includes selecting a size and/or shape of the container and/or of compartment/s of the container. For example, based on the size and/or shape of the medication of the prescription.

In some embodiments, selection of a container includes forming at least a portion of the container. In some embodiments, material is formed into a container e.g. by a medication dispenser (e.g. medication dispenser 104 FIG. 1). Referring back to FIG. 1, in some embodiments, medication dispenser 104 constructs containers, for example, using construction materials 116 e.g. paper and/or card from which containers 110 are constructed e.g. by folding and/or cutting and/or connecting (e.g. sticking). In some embodiments, construction material 116 includes different sizes of material e.g. different sheet sizes of paper and/or card e.g. for forming different sized containers. In some embodiments, construction material 116 includes one or more of fabric, plastic, metal, paper, and cardboard.

In some embodiments, containers are envelopes constructed by the system e.g. where sheet material (e.g. paper, card, sheet plastic) loaded to the system is constructed into containers (e.g. by folding and/or sealing and/or sticking).

In some embodiments, medication doses are dispensed sequentially according to prescribed timing, for example, all morning doses for all patients are dispensed and only then are afternoon doses dispensed. In some embodiments, medication doses are dispensed by patient, for a time period. For example, a week of doses (or other time period, e.g. a day, a week, a month) for an individual patient are dispensed before the medication dispenser dispenses for the next patient.

In some embodiments, for example, using a container including multiple medication doses for a specific patient requires less delivery time resources as separate doses do not need to be separately delivered. It puts less time restrictions on the medication dispenser (a dispensing resource) as the medication dispenser has a single time requirement. However, medication dispenser time is occupied with dispensing for a single patient, for a longer time.

Exemplary different container configurations include, in some embodiments, different types of container (e.g. different number of compartments) and different uses of the same container. For example, in some embodiments, a multi-compartment container is used to supply more than one patient (and/or group of patients), different compartments designated for different patients (and/or groups of patients).

In some embodiments, the calculating is for a plurality of medication dispensers.

At 612, in some embodiments, (e.g. based on calculation/s as described in step 610) an optimal container and/or distribution configuration is selected.

At 614, in some embodiments, containers are filled with the appropriate medications, e.g. where filling includes one or more feature as described regarding medication dispenser 204 FIG. 2A.

In some embodiments, a container is filled with medication dose/s for a particular patient. Where, in some embodiments, one or more dose includes different medication types to be taken during a time period.

At 616, in some embodiments, medication containers are delivered to patients and/or patient spaces e.g. on a delivery vehicle (e.g. delivery vehicle 220 FIG. 2A).

At 618, optionally, in some embodiments, compartment/s of the medication container are separated. For example, separation of compartment/s each compartment including one or more medication dose. Alternatively or additionally, in some embodiments, sub-containers are removed from the medication container.

At 620, in some embodiments, after separation of compartment/s the compartments are delivered and/or sub-containers are delivered.

For example, in some embodiments, a container includes multiple compartments, each compartment containing medication for a patient. The compartments are then separated for separate delivery to individual patients.

At 622, in some embodiments, medication from more than one compartment is mixed e.g. prior to dispensing. For example, where mixing includes one or more feature as described regarding FIGS. 15A-B. In some embodiments, step 622 occurs after delivery verification and/or after verification of a particular time for the mixing e.g. in embodiments where mixing is immediately (or close) to medication dispensing and/or administration.

At 624, delivery of the medication container is verified.

In some embodiments, verification of delivery includes verifying that the medication container is physically within a patient space and/or in proximity to the patient. In some embodiments, this is monitored e.g. verification of delivery is repeated, e.g. during a dispensing time period accounted for with doses of medication within the medication container. In some embodiments, verifying includes a particular proximity to the patient, optionally, with an allowed threshold time period when the patient and medication container are separated beyond the particular proximity. Where, in some embodiments, the particular proximity is the same care facility or same building or same room as the patient or within 50 m, or 20 m, or lower or higher or intermediate distances, of the patient.

At 626, in some embodiments, if delivery is verified, step 630 is initiated.

At 628, in some embodiments, if delivery verification fails an alert is initiated, for example, an alert signal is sent to one or more controller (e.g. medication container controller, e.g. medication dispenser controller). In some embodiments, the alert is displayed on one or more user interface e.g. medication container user interface and/or medication dispenser user interface.

At 630, in some embodiments, delivery verification data is stored and/or transmitted, for example, by the medication container and/or the medication dispenser. For example, in some embodiments, the medication container sends the verification data to the medication dispenser. For example, data acquired by one or more container sensor and/or data inputted at one or more container user interface. In some embodiments, verification data is stored in the patient's medical record e.g. electronic medical record which, in some embodiments, is hosted by the medication dispenser.

At 632, in some embodiments, the patient opens the medication container.

At 634, in some embodiments, opening of the medication container is verified, for example, using one or more sensor signal and/or one or more signal received at a user interface. For example, in some embodiments, the medication container includes one or more sensor configured to sense opening of the medication container, and in some embodiments, to sense opening of individual compartment/s of the medication container. In some embodiments, a user (e.g. patient and/or caregiver) enters an input into the user interface that the container has been opened.

At 636, if verification of opening fails an alert is initiated e.g. including one or more feature as described regarding step 628.

At 638, in some embodiments, if verification of opening of the medication container fails, a caregiver opens the container and/or assists the patient in opening the container. In some embodiments, the caregiver, having been alerted at step 636 is instructed to assist the patient in opening the medication.

In some embodiments, for example, alternatively to step/s 632-638, supervision of the patient is verified and the supervisor opens and/or assists the patient in opening the medication container.

In some embodiments, for example, alternatively to step/s 632-638, the container opens automatically (e.g. upon verifying delivery and/or appropriate supervision and/or of correct timing). Where, for example, opening is initiated by a controller which sends an opening control signal to one or more actuator of the medication container configured to open the medication container.

In some embodiments, opening verification data is stored and/or transmitted e.g. storing and/or transmission including one or more feature as described regarding step 630. Where, in some embodiments, opening verification data is received by the system e.g. by the medication dispenser e.g. as described in step 606.

At 642, in some embodiments, the patient consumes one or more medication dose which has been revealed by opening the medication container. In some embodiments, the opened portion of the container includes correct medication or medications for a time and/or time period, e.g. according to a patient prescription.

At 644, in some embodiments, medication use (e.g. consumption) is verified. For example, by a caregiver supervising the patient where the caregiver then enters an input into a user interface indicating that the medicine was consumed. In some embodiments, one or more sensor signal is used to validate medication use (e.g. consumption). For example, one or more sensor of the medication container and/or one or more sensor in the patient space.

In some embodiments, medication use is verified remotely. For example, in some embodiments, sensor signals are evaluated (e.g. by a care giver) remotely. For example, in some embodiments optical sensor signals (e.g. photograph and/or video footage collected by one or more camera).

At 646, if medication use (e.g. consumption) is verified, step 650 is initiated.

At 648, in some embodiments, if medication use (e.g. consumption) verification fails, an alert is initiated, for example, an alert signal is sent to one or more controller (e.g. medication container controller, e.g. medication dispenser controller). In some embodiments, the alert is displayed on one or more user interface e.g. medication container user interface and/or medication dispenser user interface.

At 650, in some embodiments, medication use verification data is stored and/or transmitted, for example, by the medication container and/or the medication dispenser. For example, in some embodiments, the medication container sends the verification data to the medication dispenser. For example, data acquired by one or more container sensor and/or data inputted at one or more container user interface. In some embodiments, verification data is stored in the patient's medical record e.g. electronic medical record which, in some embodiments, is hosted by the medication dispenser.

At 651, in some embodiments, an action is performed upon receipt (e.g. by the medication dispenser) of medication verification data (e.g. from one or more of medication container delivery verification, medication container opening verification, supervision verification and medication use verification).

For example, in some embodiments, patient medical record/s are updated with verification/s. In some embodiments, the updated record/s are analyzed and further action/s are performed, based on the analysis. For example, if it is determined that a self-administering patient is not successfully self-administering medication, a patient status is changed to “requiring assistance”. For example, if it is determined that a patient missed one or more dose, procedure with respect to the next medication dose is changed. For example, instructions are issued to administer a subsequent dose at an earlier time and/or to substitute a different medication. Alternatively or additionally, a missed dose may generate an alert that the patient requires monitoring. For example, if it is determined that a compartment and/or sub-container has not been opened, medication therein is considered as “not dispensed” enabling the medication, for example, to be used at a different time and/or transferred (e.g. back to the medication dispenser).

At 652, in some embodiments, the medication container is collected and/or returned. For example, for retrieval of medication container data. For example, for re-use.

At 654, in some embodiments, the container is sterilized e.g. prior to re-use.

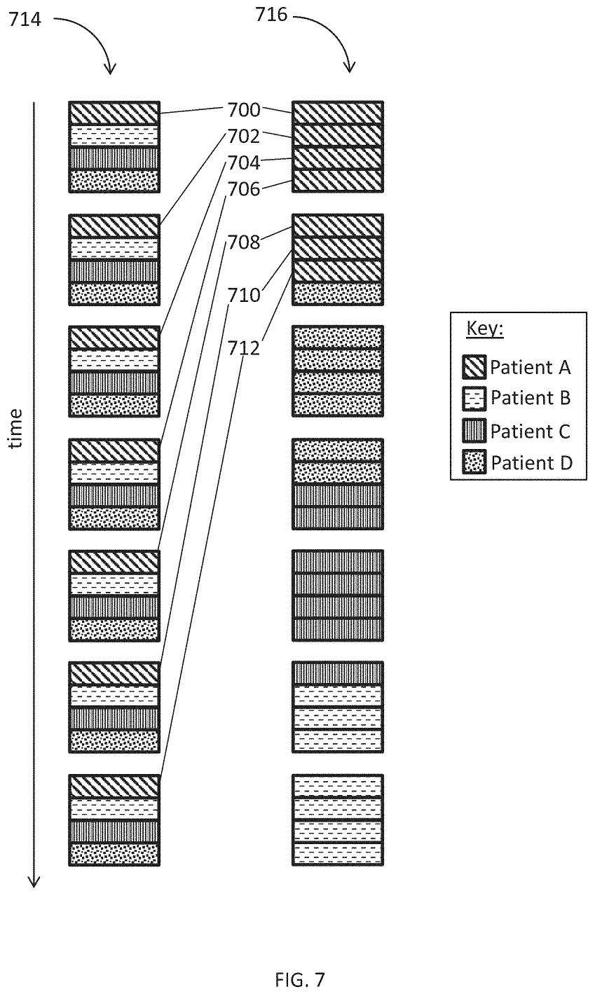

FIG. 7 illustrates two dispensing schedules 714, 716, according to some embodiments of the invention.

In some embodiments, each of 700-712 represent a daily dose for a particular patient. Where different shading represents a dose for a different patient (patients, A-C as illustrated by the “key” of FIG. 7).

In some embodiments, dispensing schedule 714 illustrates a dispensing schedule where four patients A-C have daily medication dispensed, each day.

In some embodiments, dispensing schedule 716 illustrates a dispensing schedule where each patient has a week worth of medication sequentially dispensed e.g. into a multiple-compartment container. A potential advantage of the dispensing schedule of 716 is that it enables delivery of dispensed medication to the patient at a lower frequency e.g. once a week.

In some embodiments, FIG. 7 illustrates different time scales. For example, in some embodiments, FIG. 7 illustrates shorter time scales, for example, where patients require medicating 7 times a day, where dispensing schedule 716 enables delivering dispensed medication once a day.

### Exemplary Multi-Compartment Container

FIGS. 8A-8D are simplified schematics illustrating exemplary use of a medication container 810, according to some embodiments of the invention.

In some embodiments, medication container 810 includes a plurality of medication compartments 878.

In some embodiments, medication compartments 878 are closable, e.g. able to be closed and/or sealed so that, upon transportation of medication container 810, medication within compartments 878 does not fall out of medication container 810. In some embodiments, closing of the compartment/s includes sealing (e.g. by sticking and/or using a seal e.g. O-ring) e.g. potentially reducing interaction of the medication with the outside environment.

Optionally, in some embodiments, medication container 810 includes one or more user interface 860. Where user interface 860, in some embodiments, includes one or more feature as illustrated in and/or described regarding user interface 160 FIG. 1.

In some embodiments, different compartments 878 are used for different doses of medication for a specific patient, for example, different compartments for different times of the day, and/or day/s of the week. In some embodiments, different compartments 878 are used for different types of medication. In some embodiments, one or more compartment optionally contains more than one dose of a medication (where, in some embodiments, instructions are issued regarding the amount of medication to be taken at a time period e.g. instructions issued by user interface 860).

FIG. 8A, in some embodiments, illustrates exemplary filling of compartments 878 of medication container 810. Where, for example, in some embodiments, medication 874 falls (e.g. under gravity) into container compartment/s.

FIG. 8B illustrates medication 874 within each compartment 878 of medication container 810.

In some embodiments, medication 874 within compartments 878 is secured within medication container 810 by one or more cover 876. In some embodiments, each compartment 878 of medication container 810 has an individual cover 876.

FIG. 8B illustrates medication container 810 where covers 876 are closed. FIG. 8D illustrates medication container 810 where a compartment 878 has been opened by moving of cover 876 (e.g. to access medication 874).

In some embodiments, one or more cover 876 is hinged and opened by rotation about a hinge. Alternatively, in some embodiments, one or more cover opens by sliding e.g. along a track.

Optionally, in some embodiments, medication container 810 includes one or more actuator 858. In some embodiments, one or more compartment 878 is opened by actuator 858 e.g. by moving cover 876.

Optionally, in some embodiments, medication container includes one or more sensor 862. Where sensor/s 862, in some embodiments, sense one or more of opening of compartment/s, removal of medication, information regarding the patient and/or patient environment.

Optionally, in some embodiments, medication container 810 includes a controller 856. In some embodiments, controller 856 includes feature/s of controller 156 FIG. 1. In some embodiments, controller 856 controls actuator/s 858. In some embodiments, controller 856 saves and/or transmits verification data.

In some embodiments, medication container 810 includes a position tracker (e.g. accelerometer and/or GPS). In some embodiments, medication container 810 includes a transmitter and/or receiver e.g. for communicating a signal e.g. a sensor signal. In some embodiments, medication container 810 includes a memory (e.g. including feature/s of memory 153 FIG. 1).

In some embodiments, the memory stores one or more of; information regarding the medication within the container (e.g. instructions for use of the medication), information regarding the patient's prescription, individual patient details, location of the patient and/or patient environment. In some embodiments, the memory communicates with one or more external device (e.g. medication dispenser 204 FIG. 2A and/or base 296 FIG. 2B-C) for example, for display and/or adjustment of the stored information.

FIG. 9 is a simplified schematic of a medication container 911, according to some embodiments of the invention.

In some embodiments, medication container 911 includes one or more identifier 946, 947. Where identifier/s include one or more feature as illustrated and/or described regarding identifier 246 FIGS. 2A-C.

In some embodiments, identifier 946 is printed onto and/or adhered to medication container 911. In some embodiments, identifier 947 is held in and/or coupled to medication container 911 by an identifier compartment 949.

In some embodiments, one or more identifier 946, 947 change/s and/or generates a signal when the medication container 910 is opened. Where, in some embodiments, identifiers include RFIDs where, in some embodiments, upon opening one or more RFID changes signal. For example, in some embodiments, an identifier (e.g. RFID) is configured to be broken (e.g. torn) when medication container 911 is opened.

In some embodiments, medication container 911 includes one or more impedance sensor where the sensor signal changes (e.g. measured impedance changes) upon opening of the medication container e.g. opening and/or tearing of the envelope.

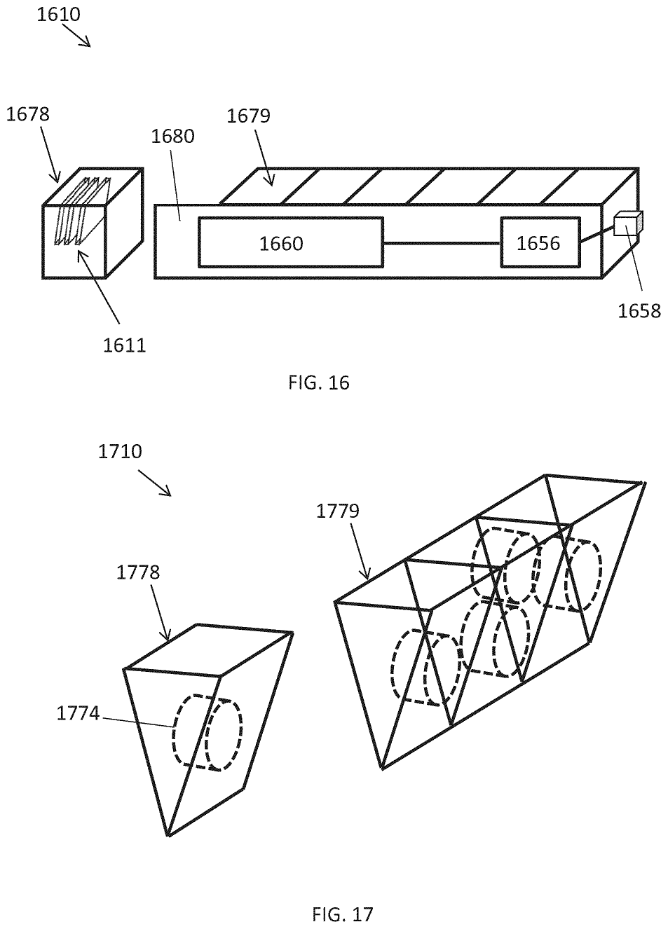

In some embodiments, medication container 911 is a sub-container configured to be placed into a compartment of a medication container (e.g. medication containers 110 FIG. 1, 210 FIGS. 2A-C, 810 FIGS. 8A-D, 1010 FIG. 10, 1111 FIG. 11, 1210 FIG. 12, 1311 FIGS. 13A-C, 1410 FIG. 14, 1510 FIGS. 15A-B, 1610 FIG. 16, 1710 FIG. 17.

In some cases, the container is a paper envelope 911 that has one or more opening tabs. Opening, in some embodiments, is from one or more side of the envelope and/or from one or more corner of the envelope. In some embodiments, medication container 911 is closed by crimping and/or adhesion.

In some embodiments, medication container 911 is disposable, for example, is a paper envelope, for example, is a single use plastic box, for example, the medication container is designed to be destroyed by opening.

In some embodiments, medication container 911 includes one or more disposable portion and one or more reusable portion.

In some embodiments, medication container 911 includes one or more electronics unit 947. In some embodiments, a body of medication container 911 is disposable (e.g. is an envelope) and, in some embodiments, electronics unit 947 is re-usable. For example, is removed and returned to the medication dispenser e.g. for re-use.

In some embodiments, electronics unit 947 includes one or more sensor e.g. the sensor/s configured to sense opening and/or movement of the medication container 911. In some embodiments, electronics unit 947 includes a position tracker (e.g. accelerometer and/or GPS). In some embodiments, electronics unit 947 includes a transmitter and/or receiver e.g. for communicating a signal e.g. sensor signal. In some embodiments, electronics unit 947 includes a memory and/or controller (e.g. including feature/s of memory 153 and/or controller 156 of FIG. 1 respectfully).

In some embodiments, a memory (e.g. hosted by electronics unit 947) stores one or more of; information regarding the medication within the container (e.g. instructions for use of the medication), information regarding the patient's prescription, individual patient details, location of the patient and/or patient environment. In some embodiments, the memory communicates with one or more external device (e.g. medication dispenser 204 FIG. 2A and/or base 296 FIG. 2B-C) for example, for display and/or adjustment of the stored information.

FIG. 10 is a simplified schematic of a medication container 1010, according to some embodiments of the invention.

In some embodiments, medication container 1010 is used to hold sub-containers 1011. In some embodiments, a multiple compartment 1078 medication container is configured to hold a sub-container 1011 within each compartment 1078. In some embodiments, insertion of sub-container 1011 into a compartment 1078 is laterally, e.g. as illustrated by arrow 1019. In some embodiments, insertion is using the same actuator/s and/or is in the same direction as movements when filling sub-container 1011 with medication e.g. movements 118 as illustrated in FIG. 1. In some embodiments, sub-container 1011 is a folded container e.g. an envelope.

FIG. 11 is a simplified schematic of a medication container 1110, according to some embodiments of the invention.

In some embodiments, medication container 1110 includes a plurality of compartments 1178 where each compartment 1178 is configured (e.g. sized and/or shaped and/or configured to sense presence of) to hold a single sub-container 1111.

FIG. 12 is a simplified schematic of a medication container 1210, according to some embodiments of the invention.

In some embodiments, medication container 1210 includes a plurality of compartments 1278 where each compartment 1278 is configured (e.g. sized and/or shaped and/or configured to sense presence of) to hold a plurality of sub-containers 1211. For example, 2-10 sub-containers. For example, 7 sub-containers e.g. for a weeks' worth of medication.

FIGS. 13A-C are simplified schematics of a medication container 1311, according to some embodiments of the invention.

In some embodiments, medication container 1311 is a medication sub-container for placing into another medication container and/or into a compartment of another medication container.

In some embodiments, medication container 1311 is includes (e.g. is constructed of) flexible and/or tearable material (e.g. paper). FIG. 13A in some embodiments illustrates filling of medication container 1311. FIG. 13B, in some embodiments, illustrates a filled and closed container 1311. FIG. 13C, in some embodiments, illustrates opening of a compartment 1378 by unsticking and/or tearing, for example, of cover 1376 and/or another part of a body of medication container 1380. In some embodiments, opening of compartment 1378 includes separating compartment 1378 from medication container 1311.

FIG. 14 is simplified schematic of a medication container 1410, according to some embodiments of the invention.

In some embodiments, medication container 1410 includes a compartment 1478 and optionally, in some embodiments, includes a plurality of compartments 1478.

In some embodiments, one or more compartment 1478 has more than one opening 1482, 1484. In some embodiments, medication 1474 is placed into compartment 1478 through a first opening 1482 and is dispensed from compartment 1478 through a second opening 1484. In some embodiments, medication container 1410 is orientated so that medication 1474 is placed into compartment 1478 and/or dispensed 1475 from compartment 1478 under gravity. In some embodiments, one or more of openings 1476, 1486 are closed using one or more cover 1476, 1484 e.g. a cover for each opening.

In some embodiments, a container base (e.g. base 296 FIGS. 2B-C) includes opening/s including one or more feature as described regarding medication container 1410 where, in some embodiments, medication sub-container/s are inserted into the compartments. In some embodiments, a container base (e.g. base 296, FIGS. 2B-C) includes one compartment as described regarding compartment 1478 which, in some embodiments, houses a medication container or sub-container.

FIGS. 15A-B are simplified schematics of a multi-compartment medication container 1510, according to some embodiments of the invention.

In some embodiments, medication container 1510 includes a plurality of compartments 1578 where one or more of the compartments 1578 is configured (e.g. sealable) to contain powder 1588 and/or liquid 1590.

In some embodiments, medication container 1510 includes a plurality of compartments, where the contents of at least two compartments are mixed after the medications have been placed into the compartments e.g. mixing prior to medication delivery and/or administration.

In some embodiments, medication container 1510 includes one or more mixing compartment 1594. In some embodiments, mixing compartment 1594 is connectable to one or more other compartment 1578 e.g. by compartment closure/s 1586. In some embodiments, mixing compartment 1594 is initially empty (e.g. after filling of medication container 1510) and then medications are mixed within mixing compartment 1594. In some embodiments, mixing compartment 1594 is filled during filling of medication container 1510 by a medication dispenser, and then medication from one or more other compartment is placed (e.g. allowed to enter into mixing compartment 1594) and then optionally mixed with the medication in mixing department 1594.

In some embodiments, a plurality of medication compartments are opened into mixing compartment 1594 allowing (e.g. under gravity) flow of contents of the medication compartments into mixing compartment 1594. In some embodiments, the contents are agitated to mix e.g. by one or more actuator (not illustrated) and/or by a user e.g. manual shaking medication container 1510.

FIG. 16 is a simplified schematic of a multi-compartment 1679 medication container 1610, according to some embodiments of the invention.

In some embodiments, one or more compartment 1678 is detachable from other portion/s of medication container 1610. For example, in some embodiment, medication container 1610 carries medication for a plurality of patients and compartment/s are detached and delivered separately to different patients and/or patient groups.

In some embodiments, FIG. 16 illustrates embodiments where each compartment holds multiple medication doses (and/or medication sub-containers 1611) the doses and/or sub-containers e.g. for an individual patient at different times.

FIG. 17 is a simplified schematic of a multi-compartment 1779 medication container 1710, according to some embodiments of the invention.

In some embodiments, one or more compartment 1778 is detachable from other portion/s of medication container 1710.

In some embodiments, FIG. 17 illustrates embodiments where each compartment holds a single dose 1774 (e.g. for a specific patient at a specific time). Where, in some embodiments, a single dose includes more than one medication type and/or medication unit (e.g. pill).

### General

It is expected that during the life of a patent maturing from this application many relevant medication dispensing technologies and/or medication containers will be developed and the scope of the term medication dispensing and/or medication container is intended to include all such new technologies a priori.

As used herein the term “about” refers to ±20%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of dispensing medication comprising:
filling a medication container with one or more medication dose, using one or more actuator of a medication dispenser device, according to a prescription for a specific patient, where said prescription is stored in a patient record for said specific patient;
verifying delivery of said medication container to a vicinity of said specific patient and/or an environment of said specific patient, where said verifying includes generating a delivery verification signal;
verifying use of said one or more medication dose, where said verifying includes generating a use verification signal; and
where one or both of said use verification signal and said delivery verification signal are generated using and/or by interacting with said medication container.

2. The method according to claim 1, comprising updating said patient record, using said delivery verification signal, and said use verification signal.

3. The method according to claim 2, comprising evaluating a patient compliance, for a time period, by comparing said use verification signal and said prescription;
wherein said updating comprises updating said patient record using said patient compliance.

4. The method according to claim 3, wherein said one or more medication dose includes medication, according to said prescription, for 1-31 time periods.

5. The method according to claim 3, wherein said one or more medication dose includes medication, according to said prescription, for 1-7 time periods.

6. The method according to claim 1, comprising evaluating one or more of said delivery verification signal and said use verification signal; and
performing an action, based on said evaluating.

7. The method according to claim 1, wherein said verifying use comprises verifying opening of said medication container.

8. The method according to claim 1, wherein said verifying use comprises verifying removal of medication from said medication container.

9. The method according to claim 1, comprising generating a container opening signal, upon opening of said medication container.

10. The method according to claim 1, wherein said verifying use comprises receiving and evaluating an input at a user interface.

11. The method according to claim 1, wherein said verifying delivery comprises sensing and evaluating said sensing.

12. The method according to claim 1, wherein said verifying delivery comprises receiving and evaluating an input at a user interface.

13. The method according to claim 1, wherein said verifying delivery of said medication container to said specific patient comprises matching a medication container identifier to a patient identifier, using identifier records stored by said medication dispenser.

14. The method according to claim 1, wherein said verifying delivery includes evaluating GPS tracking signal.

15. A medication container comprising:
at least one closeable compartment configured to store at least one dose of medication;
at least one medication container identifier;
one or more element configured to be used and/or interacted with in generation of one or both of:
a delivery verification feedback signal, upon delivery of said medication container to a patient vicinity and/or environment; and
a use verification feedback signal, upon use of medication held within said at least one closeable compartment.

16. The medication container of claim 15, comprising a memory configured to store one or more of patient information and medication information.

17. The medication container of claim 15, wherein said one or more element includes a position tracker, where position of said medication container is used to generate said delivery verification feedback signal.

18. The medication container of claim 15, comprising one or more sensor configured to sense an indication of use of said at least one dose of medication.

19. The medication container of claim 18, wherein said one or more sensor includes a sensor configured to sense opening of said at least one closeable compartment.

20. The medication container of claim 19, wherein said sensor configured to sense opening of said at least one closeable compartment includes an impedance sensor.

21. The medication container of claim 20, wherein said one or more sensor includes a sensor configured to sense removal of medication from said at least one closeable compartment.

22. The medication container of claim 21, wherein said a sensor configured to sense removal of medication from said at least one closeable compartment is a weight sensor.

23. The medication container of claim 15, comprising a disposable housing and a reusable electronics unit.

24. The medication container of claim 15, comprising a plurality of closeable compartments.

25. The medication container of claim 15, wherein said medication container identifier is used to generate said delivery verification feedback signal.
